(19) United States
(12) Patent Application Publication
(10) Pub. No.: US 2024/0396083 A1
(43) Pub. Date: Nov. 28, 2024

(54) ELECTROLYTE ADDITIVES FOR RECHARGEABLE LITHIUM BATTERY, ELECTROLYTE INCLUDING THE SAME, AND RECHARGEABLE LITHIUM BATTERY INCLUDING THE SAME

(71) Applicant: SAMSUNG SDI CO., LTD., Yongin-si (KR)

(72) Inventors: Yeji YANG, Yongin-si (KR); Sundae KIM, Yongin-si (KR); Hyunbong CHOI, Yongin-si (KR); Sangwoo PARK, Yongin-si (KR); Dahyun KIM, Yongin-si (KR); Hongryeol PARK, Yongin-si (KR); Sanghoon KIM, Yongin-si (KR)

(21) Appl. No.: 18/494,255

(22) Filed: Oct. 25, 2023

(30) Foreign Application Priority Data
May 23, 2023 (KR) 10-2023-0066538

(51) Int. Cl. H01M 10/0567; C07D 403/06; H01M 4/505; H01M 4/525; H01M 10/052; H01M 10/0569; H01M 10/44

(52) U.S. Cl. CPC H01M 10/0567; C07D 403/06; H01M 4/505; H01M 4/525; H01M 10/052; H01M 10/0569; H01M 10/44; H01M 2004/028; H01M 2300/0028

(57) ABSTRACT

An electrolyte additive for a rechargeable lithium battery, an electrolyte including the electrolyte additive, and a rechargeable lithium battery including the electrolyte are provided. The electrolyte additive for a rechargeable lithium battery includes an additive represented by Chemical Formula 1:

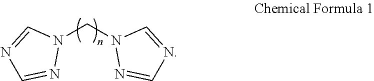

### CROSS-REFERENCE TO RELATED APPLICATION

The present application claims priority to and the benefit of Korean Patent Application No. 10-2023-0066538, filed on May 23, 2023, in the Korean Intellectual Property Office, the entire content of which is incorporated herein by reference.

### BACKGROUND

### 1. Field

One or more embodiments of the present disclosure relate to electrolyte additives for a rechargeable lithium battery, electrolytes, and rechargeable lithium batteries.

### 2. Description of Related Art

A rechargeable lithium battery may be recharged and has three or more times as high (i.e., has at least three times more) energy density per unit weight as compared to a comparable lead storage battery, nickel-cadmium battery, nickel hydrogen battery, nickel zinc battery, and/or the like. The rechargeable lithium battery may be also charged at a relatively high rate and thus, is commercially manufactured for a laptop, a cell phone, an electric tool, an electric bike, and/or the like. Also, research on improvement of additional energy density has been actively made and/or pursued.

An example rechargeable lithium battery is made by injecting an electrolyte into an electrode assembly including a positive electrode including a positive electrode active material and a negative electrode including a negative electrode active material.

One of the recent development directions for a rechargeable lithium battery is to improve high-temperature charge/discharge and storage characteristics while also improving room temperature charge/discharge characteristics. In general, a rechargeable lithium battery has a problem in that its resistance increases and/or its cycle-life decreases as it is repeatedly charged and discharged at room temperature. This problem becomes more severe when the rechargeable lithium battery is stored at high temperatures.

### SUMMARY

One or more aspects of embodiments of the present disclosure are directed toward an electrolyte additive for a rechargeable lithium battery that improves high-temperature charge/discharge and storage characteristics while also improving room temperature charge/discharge characteristics of the rechargeable lithium battery.

Additional aspects will be set forth in part in the description which follows and, in part, will be apparent from the description, or may be learned by practice of the presented embodiments of the disclosure.

According to one or more embodiments of the present disclosure, an electrolyte additive for a rechargeable lithium battery includes an additive represented by Chemical Formula 1:

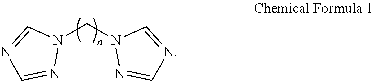

In Chemical Formula 1, n is an integer from 1 to 6.

The electrolyte additive for a rechargeable lithium battery according to one or more embodiments may improve high temperature charge/discharge and storage characteristics while also improving room temperature charge/discharge characteristics of the rechargeable lithium battery.

### BRIEF DESCRIPTION OF THE DRAWINGS

The accompanying drawing is included to provide a further understanding of the present disclosure, and is incorporated in and constitutes a part of this specification. The drawing illustrates example embodiments of the present disclosure and, together with the description, serve to explain principles of present disclosure. In the drawing:

FIGS. 1-3 are each of schematic views showing rechargeable lithium batteries according to one or more embodiments of the present disclosure.

### DETAILED DESCRIPTION

The present disclosure may be modified in many alternate forms, and thus specific embodiments will be exemplified in the drawing and described in more detail. It should be understood, however, that it is not intended to limit the present disclosure to the particular forms disclosed, but rather, is intended to cover all modifications, equivalents, and alternatives falling within the spirit and scope of the present disclosure.

Hereinafter, a rechargeable lithium battery according to one or more embodiments will be described in more detail with reference to the accompanying drawings. However, these embodiments are mere examples, the present disclosure is not limited thereto, and the present disclosure is defined by the scope of claims and equivalents thereof.

As previously described, one of the recent development directions for rechargeable lithium batteries is to improve high-temperature charge/discharge and storage characteristics while also improving room temperature charge/discharge characteristics. In general, a rechargeable lithium battery has a problem in that its resistance increases and/or its cycle-life decreases as it is repeatedly charged and discharged at room temperature. This problem becomes more severe when the rechargeable lithium battery is stored at high temperatures.

In order to solve the above problem, a method of adding an additive to the electrolyte of the rechargeable lithium battery has been proposed and/or pursued, but additives proposed to date are insufficient to solve the above problem. For example, for a benzotriazole-based additive, the benzene ring in the structure thereof may be oxidized and decomposed on the surface of the positive electrode of the rechargeable lithium battery to form an unstable film, increasing a resistance of the rechargeable lithium battery and reducing its cycle-life. This phenomenon becomes more noticeable when the rechargeable lithium battery is stored at high temperatures.

In one or more embodiments of the present disclosure, a compound including triazole itself without a fused benzene ring is utilized as an additive to improve high temperature charge/discharge and storage characteristics of a rechargeable lithium battery while also improving room temperature charge/discharge characteristics. Unlike a benzotriazole-based additive, the compound including triazole itself without a fused benzene ring maintain a stable structure not only at room temperature but also when stored at high temperatures. As a result, the compound including triazole itself without a fused benzene ring may produce a chain effect of adsorbing on the positive electrode active material, suppressing elution of metal from the positive electrode active material, and suppressing an increase in resistance of the rechargeable lithium battery.

Hereinafter, a detailed description of some example embodiments will be disclosed.

### Electrolyte Additives

One or more embodiments provide an electrolyte additive for a rechargeable lithium battery, including an additive represented by Chemical Formula 1:

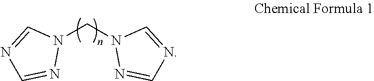

In Chemical Formula 1, n may be an integer from 1 to 6.

The additive in one or more embodiments may be a dimer in which two triazoles are linked by an alkylene linking group. As the alkylene linking group, an alkylene linking group having 1 to 6 carbon atoms without any functional group (i.e., an unsubstituted alkylene group) may be utilized. Herein, when two triazoles are linked by an alkylene linking group with more than 6 carbon atoms, a resistance of rechargeable lithium battery increases, whereas when they are linked by a single bond, they may function like a monomer rather than a dimer (i.e., only one-triazole functions).

In some embodiments, an alkylene having 4, 5, or 6 carbon atoms may be utilized as the alkylene linking group. For example, in Chemical Formula 1, n may be an integer of 4 to 6.

Representative examples of additives represented by Chemical Formula 1 may be as follows:

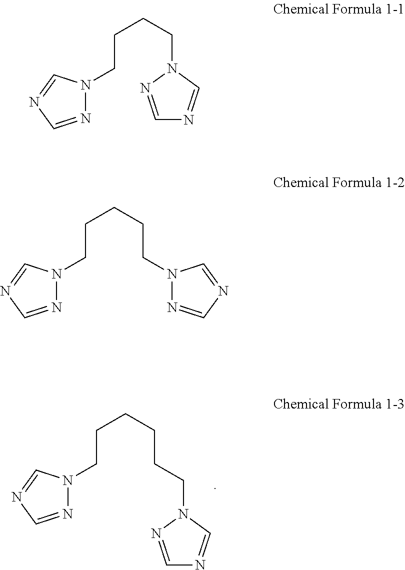 [SMILES: c1ncn(CCCCn2cncn2)n1 ; c1ncn(CCCCCn2cncn2)n1 ; c1ncn(CCCCCCn2cncn2)n1]

### Electrolyte

According to one or more embodiments, an electrolyte for a rechargeable lithium battery may include a non-aqueous organic solvent; a lithium salt; and an additive represented by represented by Chemical Formula 1:

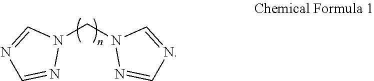

The description of Chemical Formula 1 is the same as described above.

The electrolyte of one or more embodiments may improve room temperature charge/discharge characteristics of a rechargeable lithium battery while also improving the high temperature charge/discharge and storage characteristics by including the additive of one or more embodiments.

Hereinafter, descriptions that overlap with the aforementioned content will not be provided for conciseness, and the electrolyte of one or more embodiments will be disclosed in more detail.

### Content of Additive Represented by Chemical Formula 1

The additive represented by Chemical Formula 1 may be included in an amount of about 0.05 wt % to about 3.0 wt %, about 0.1 wt % to about 1.0 wt %, or about 0.1 wt % to about 0.5 wt %, based on a total amount of the electrolyte.

If (e.g., when) an excessive amount of the additive represented by Chemical Formula 1 is included, a side reaction may occur, and resistance of the rechargeable lithium battery may increase, thereby reducing a performance of the rechargeable lithium battery. On the other hand, if (e.g., when) the additive represented by Chemical Formula 1 is included below the range described above, the effect may be minimal.

### Non-aqueous Organic Solvent

The non-aqueous organic solvent serves as a medium for transmitting ions taking part in the electrochemical reaction of a battery.

The non-aqueous organic solvent may be a carbonate-based, ester-based, ether-based, ketone-based, or alcohol-based solvent, an aprotic solvent, or a combination thereof.

The carbonate-based solvent may include dimethyl carbonate (DMC), diethyl carbonate (DEC), dipropyl carbonate (DPC), methylpropyl carbonate (MPC), ethylpropyl carbonate (EPC), ethylmethyl carbonate (EMC), ethylene carbonate. (EC), propylene carbonate (PC), butylene carbonate (BC), and/or the like. The ester-based solvent may include methyl acetate, ethyl acetate, n-propyl acetate, dimethyl acetate, methyl propionate, ethyl propionate, decanolide, mevalonolactone, valerolactone, caprolactone, and/or the like. The ether-based solvents may include dibutyl ether, tetraglyme, diglyme, dimethoxyethane, 2-methyltetrahydrofuran, 2,5-dimethyltetrahydrofuran, tetrahydrofuran, and/or the like. Additionally, the ketone-based solvent may be cyclohexanone, and/or the like. The alcohol-based solvent may be ethyl alcohol, isopropyl alcohol, and/or the like, and the aprotic solvent may be nitriles such as R—CN (wherein R is a linear, branched, or cyclic hydrocarbon group having 2 to 20 carbon atoms, and may include a double bond, an aromatic ring, or an ether group); amides such as dimethylformamide; dioxolanes such as 1,3-dioxolane and 1,4-dioxolane; sulfolanes; and/or the like.

The non-aqueous organic solvent may be utilized alone or in a mixture of two or more types (kinds), and if (e.g., when) two or more types (kinds) are utilized in a mixture, a mixing ratio may be appropriately adjusted according to the desired or suitable battery performance, and this should be understood by those working in the field.

In some embodiments, if (e.g., when) a carbonate-based solvent is utilized, it is desirable to utilize a chain carbonate alone, excluding cyclic carbonate (cyclic carbonate-free). But, in some embodiments, a mixture of cyclic carbonate and chain carbonate may be utilized, and in these embodiments, cyclic carbonate and chain carbonate may be mixed at a volume ratio of about 1:1 to about 1:9. For example, in some embodiments, the non-aqueous organic solvent may include ethylene carbonate (EC), ethylmethyl carbonate (EMC), dimethyl carbonate (DMC), or a combination thereof. In present disclosure, “not including a or any ‘component” “excluding a or any ‘component”, “component’-free”, and/or the like refers to that the “component” not being added, selected, or utilized as a component in the composition, but the “component” of less than a suitable amount may still be included due to other impurities and/or external factors.

In one or more embodiments, the non-aqueous organic solvent may further include an aromatic hydrocarbon-based organic solvent. For example, in some embodiments, a carbonate-based solvent and an aromatic hydrocarbon-based organic solvent may be mixed and utilized in a volume ratio of about 1:1 to about 30:1.

### Lithium Salt

The lithium salt dissolved in the organic solvent supplies lithium ions in a battery, enables a basic operation of a rechargeable lithium battery, and improves transportation of the lithium ions between positive and negative electrodes of the rechargeable lithium battery. Representative examples of the lithium salt may include one or more selected from LiPF₆, LiBF₄, LiSbF₆, LiAsF₆, LiClO₄, LIAIO₂, LiAlCl₄, LiPO₂F₂, LiCl, Lil, LiN(SO₃C₂F₅)₂, Li(FSO₂)₂N (lithium bis(fluorosulfonyl)imide (LiFSI)), LiC₄F₉SO₃, LiN(CₓF₂ₓ₊₁SO₂) (C_{y}F_{2y+1}SO₂) (wherein x and y are each independently an integer from 1 to 20), lithium trifluoromethane sulfonate, lithium tetrafluoroethanesulfonate, lithium difluorobis(oxalato)phosphate (LiDFOB), and lithium bis(oxalato) borate (LiBOB).

The lithium salt may be utilized in a concentration in a range of about 0.1 M to about 2.0 M. When the concentration of the lithium salt is within the above range, the electrolyte has appropriate or suitable ion conductivity and viscosity, and thus excellent or suitable performance may be achieved and lithium ions may move effectively.

### Additional Additives

In one or more embodiments, the electrolyte may further include vinylethylene carbonate, vinylene carbonate, and/or ethylene carbonate-based compounds to improve cycle-life of a battery.

Representative examples of the ethylene carbonate-based compound may include fluoroethylene carbonate, difluoroethylene carbonate, chloroethylene carbonate, dichloroethylene carbonate, bromoethylene carbonate, dibromoethylene carbonate, nitroethylene carbonate, and cyanoethylene carbonate.

### Rechargeable Lithium Battery

In one or more embodiments, a rechargeable lithium battery may include a positive electrode including a positive electrode active material; a negative electrode containing a negative electrode active material; and the electrolyte of one or more embodiments.

Because the rechargeable lithium battery includes the electrolyte of one or more embodiments, an increase in resistance may be suppressed or reduced and cycle-life may be secured even when the rechargeable lithium battery is stored at high temperature.

Hereinafter, descriptions that overlap with the aforementioned content will not be provided for conciseness, and the rechargeable lithium battery of one or more embodiments will be disclosed in more detail.

### Positive Electrode Active Material

The positive electrode active material may be a compound capable of reversibly intercalating and deintercalating lithium (a lithiated intercalation compound). For example, in some embodiments, one or more types (kinds) of composite oxides of lithium and a metal selected from among cobalt, manganese, nickel, and combinations thereof may be utilized.

The composite oxide may be a lithium transition metal composite oxide, and non-limiting examples thereof may include lithium nickel-based oxides, lithium cobalt-based oxides, lithium manganese-based oxides, lithium iron phosphate-based compounds, cobalt-free nickel-manganese-based oxides, or one or more combinations thereof.

In one or more embodiments, the positive electrode active material may be a high nickel-based positive electrode active material having a nickel content (e.g., amount) of about 80 mol % or more based on 100 mol % of metals excluding lithium in the lithium transition metal composite oxide. The nickel content (e.g., amount) in the high nickel-based positive electrode active material may be about 85 mol % or more, about 90 mol % or more, about 91 mol % or more, or about 94 mol % or more, and about 99 mol % or less based on 100 mol % of metals excluding lithium in the high nickel-based positive electrode active material. High-nickel-based positive electrode active materials may achieve high capacity and may be applied to a high-capacity, high-density rechargeable lithium battery.

In one or more embodiments, a compound represented by any of the following chemical formulas may be utilized: LiₐA_{1-b}X_{b}O_{2-c}D_{c} (0.90≤a≤1.8, 0≤b≤0.5, 0≤c≤0.05); LiₐMn_{2-b}X_{b}O_{4-c}D_{c} (0.90≤a≤1.8, 0≤b≤0.5, 0≤c≤0.05); LiₐNi_{1-b-c} CO_{b}X_{C}O₂₋ₐDₐ (0.90≤a≤1.8, 0≤b≤0.5, 0≤c≤0.5, 0<a<2); LiₐNi_{1-b-c}Mn_{b}X_{c}O₂₋ₐDₐ (0.90≤a≤1.8, 0≤b≤0.5, 0≤c≤0.5, 0<a<2); LiₐNi_{b}CO_{C}L¹_{d}GeO₂ (0.90≤a≤1.8, 0≤b≤0.9, 0≤c≤0.5, 0≤d≤0.5, 0≤e≤0.1); LiₐNiG_{b}O₂ (0.90≤a≤1.8, 0.001≤b≤0.1); LiₐCoG_{b}O₂ (0.90≤a≤1.8, 0.001≤b≤0.1); LiₐMn_{1-b}G_{b}O₂ (0.90≤a≤1.8, 0.001≤b≤0.1); LiₐMn₂G_{b}O₄ (0.90≤a≤1.8, 0.001≤b≤0.1); LiₐMn_{1-g}G_{g}PO₄ (0.90≤a≤1.8, 0≤g≤0.5); Li_{(3-f)}Fe₂(PO₄)₃ (0≤f≤2); LiₐFePO₄ (0.90≤a≤1.8).

In the chemical formulas, A may be nickel (Ni), cobalt (Co), manganese (Mn), or a combination thereof; X may be aluminum (Al), Ni, Co, Mn, chromium (Cr), iron (Fe), magnesium (Mg), strontium (Sr), vanadium (V), a rare earth element, or a combination thereof; D may be oxygen (O), fluorine (F), sulfur(S), phosphorus (P), or a combination thereof; G may be Al, Cr, Mn, Fe, Mg, lanthanum (La), cerium (Ce), Sr, V, or a combination thereof; Q may be titanium (Ti), molybdenum (Mo), Mn, or a combination thereof; Z may be Cr, V, Fe, scandium (Sc), yttrium (Y), or a combination thereof; and L¹ may be Mn, Al, or a combination thereof.

For example, in some embodiments, the positive electrode active material may include a cobalt-free lithium nickel manganese-based oxide.

In the present disclosure, the cobalt-free lithium nickel manganese-based oxide as a positive electrode active material refers to a positive electrode active material composed of nickel, manganese, etc. as main components without cobalt in the positive electrode active material composition.

In one or more embodiments, the cobalt-free lithium nickel manganese-based oxide may include at least one type or kind of lithium composite oxide represented by Chemical Formula 2.

LiₐNiₓMn_{y}M¹_{z}M²_{w}O_{2+b}X_{c} Chemical Formula 2

### In Chemical Formula 2,

0.5≤a<1.8,0≤b≤0.1,0≤c≤0.1,0≤w<0.1, 06≤x<1.0, 0<y<0.4, 0<z<0.1, w+x+y+Z=1,

M¹ and M² may each independently be one or more element selected from Al, Mg, Ti, zirconium (Zr), Cr, Sr, V, boron (B), tungsten (W), Mo, niobium (Nb), silicon (Si), barium (Ba), calcium (Ca), Ce, Cr, and Fe, and X may be one or more elements selected from S, F, P, and chlorine (Cl).

In some embodiments, Chemical Formula 2 may be represented by Chemical Formula 2-1.

LiₐNiₓ₁Mn_{y1}Al_{z1}M²_{w1}O_{2+b}X_{c} Chemical Formula 2-1

### In Chemical Formula 2-1,

0.5≤a<1.8, 0≤b≤0.1, 0≤c≤0.1, 0≤w1<0.1, 0.6≤x1<1.0, 0<y1<0.4, 0≤z1≤0.1, w1+x1+y1+z1=1,

M² may be one or more elements selected from Mg, Ti, Zr, Cr, Sr, V, B, W, Mo, Nb, Si, Ba, Ca, Ce, Cr, and Fe, and X may be one or more elements selected from S, F, P, and Cl.

In one or more embodiments, in Chemical Formula 2-1, 0.6≤x1≤0.9, 0.1≤y1≤0.4, and 0≤z1≤0.1, or 0.6≤x1≤0.8, 0.2≤y1≤0.4, and 0<z1≤0.1.

For example, in some embodiments, in Chemical Formula 2-1, x1 may be 0.6≤x1≤0.79, y1 may be 0.2≤y1≤0.39, and z1 may be 0.01≤z1<0.1.

### Positive Electrode

The positive electrode for a rechargeable lithium battery may include a positive electrode current collector and a positive electrode active material layer on the positive electrode current collector. The positive electrode active material layer includes a positive electrode active material and may further include a binder and/or a conductive material.

A content (e.g., amount) of the positive electrode active material may be about 90 wt % to about 99.5 wt % based on 100 wt % of the positive electrode active material layer.

In one or more embodiments, the positive electrode active material layer may further include a binder and a conductive material. Each content (e.g., amount) of the binder and the conductive material may separately be about 0.5 wt % to about 5 wt % based on 100% by weight of the positive electrode active material layer.

The binder serves to ensure that the positive electrode active material particles adhere to each other and also to adhere the positive electrode active material to the positive electrode current collector. Representative examples of the binder may include polyvinyl alcohol, carboxymethyl cellulose, hydroxypropyl cellulose, diacetyl cellulose, polyvinyl chloride, carboxylated polyvinyl chloride, polyvinyl fluoride, an ethylene oxide-containing polymer, polyvinylpyrrolidone, polyurethane, polytetrafluoroethylene, polyvinylidene fluoride, polyethylene, polypropylene, a styrene-butadiene rubber, a (meth)acrylated styrene-butadiene rubber, an epoxy resin, a (meth)acrylic resin, polyester resin, and nylon, but embodiments of the present disclosure are not limited thereto.

The conductive material is utilized to impart conductivity to the electrode, and in the battery being configured, any electronically conductive material may be utilized as long as it does not cause chemical change. Non-limiting examples of the conductive material may include a carbon-based material such as natural graphite, artificial graphite, carbon black, acetylene black, Ketjen black, a carbon fiber, a carbon nanofiber, and/or a carbon nanotube; a metal-based material including copper, nickel, aluminum, silver, etc. and in the form of a metal powder or a metal fiber; a conductive polymer such as a polyphenylene derivative; or a mixture thereof.

In some embodiments, Al may be utilized as the positive electrode current collector, but embodiments of the present disclosure are not limited thereto.

### Negative Electrode Active Material

The negative electrode active material may be a material that reversibly intercalates/deintercalates lithium ions, a lithium metal, a lithium metal alloy, a material capable of doping and dedoping lithium, and/or a transition metal oxide.

The material that reversibly intercalates/deintercalates lithium ions may be a carbon-based negative electrode active material, for example crystalline carbon, amorphous carbon, or a combination thereof. Non-limiting examples of the crystalline carbon may include graphite such as irregular, plate-shaped, flake, spherical, or fibrous natural graphite or artificial graphite, and non-limiting examples of the amorphous carbon may include soft carbon and/or hard carbon, a mesophase pitch carbonized product, calcined coke, and/or the like.

The lithium metal alloy may include an alloy including lithium and a metal selected from among sodium (Na), potassium (K), rubidium (Rb), cesium (Cs), francium (Fr), beryllium (Be), magnesium (Mg), calcium (Ca), strontium (Sr), silicon (Si), antimony (Sb), lead (Pb), indium (In), zinc (Zn), barium (Ba), radium (Ra), germanium (Ge), aluminum (Al), and tin (Sn).

A Si-based negative electrode active material or a Sn-based negative electrode active material may be utilized as a material capable of doping and dedoping lithium. The Si-based negative electrode active material may include silicon, a silicon-carbon composite, SiOₓ (0<x≤2), a Si-Q alloy (wherein Q is an element selected from among an alkali metal, an alkaline earth metal, a Group 13 element, Group 14 element (excluding Si), Group 15 element, Group 16 element, a transition metal, a rare earth element, and a combination thereof, for example Mg, Ca, Sr, Ba, Ra, Sc, Y, Ti, Zr, hafnium (Hf), rutherfordium (Rf), V, Nb, tantalum (Ta), dubnium (Db), Cr, Mo, W, seaborgium (Sg), technetium (Tc), rhenium (Re), bohrium (Bh), Fe, Pb, ruthenium (Ru), osmium (Os), hassium (Hs), rhodium (Rh), iridium (Ir), palladium (Pd), platinum (Pt), Cu, silver (Ag), gold (Au), Zn, cadmium (Cd), B, Al, gallium (Ga), Sn, In, thallium (TI), Ge, P, arsenic (As), Sb, bismuth (Bi), S, selenium (Se), tellurium (Te), polonium (Po), and a combination thereof), or a combination thereof. The Sn-based negative electrode active material may be Sn, SnO₂, a Sn alloy, or a combination thereof.

The silicon-carbon composite (e.g., in a form of particles) may be a composite of silicon and amorphous carbon. An average particle diameter (D₅₀) of the silicon-carbon composite particles may be, for example, about 0.5 μm to about 20 μm. According to one or more embodiments, the silicon-carbon composite may be in the form of silicon particles and amorphous carbon coated on the surface of the silicon particles. For example, in some embodiments, the silicon-carbon composite may include a secondary particle (core) in which silicon primary particles are assembled and an amorphous carbon coating layer (shell) on the surface of the secondary particle. The amorphous carbon may also be disposed between the silicon primary particles, and for example, the silicon primary particles may be coated with amorphous carbon. The secondary particles may exist dispersed in an amorphous carbon matrix.

In one or more embodiments, the silicon-carbon composite may further include crystalline carbon. For example, in some embodiments, the silicon-carbon composite may include a core including crystalline carbon and silicon particles and an amorphous carbon coating layer on the surface of the core. The crystalline carbon may be artificial graphite, natural graphite, or a combination thereof. The amorphous carbon may include soft carbon and/or hard carbon, a mesophase pitch carbonized product, and/or calcined coke.

If (e.g., when) the silicon-carbon composite includes silicon and amorphous carbon, a content (e.g., amount) of silicon may be about 10 wt % to about 50 wt %, and a content (e.g., amount) of amorphous carbon may be about 50 wt % to about 90 wt % based on 100 wt % of the silicon-carbon composite. In some embodiments, if (e.g., when) the silicon-carbon composite includes silicon, amorphous carbon, and crystalline carbon, a content (e.g., amount) of silicon may be about 10 wt % to about 50 wt %, and a content (e.g., amount) of crystalline carbon may be about 10 wt % to about 70 wt %, and a content (e.g., amount) of amorphous carbon may be about 20 wt % to about 40 wt % based on 100 wt % of the silicon-carbon composite.

In one or more embodiments, a thickness of the amorphous carbon coating layer may be about 5 nm to about 100 nm. An average particle diameter (D₅₀) of the silicon particles (primary particles) may be about 10 nm to about 1 μm, or about 10 nm to about 200 nm. The silicon particles may exist as silicon alone, in the form of a silicon alloy, and/or in an oxidized form. The oxidized form of silicon may be represented by SiOₓ (0<x<2), and an atomic content (e.g., amount) ratio of Si:O, which indicates a degree of oxidation, may be about 99:1 to about 33:67. In the present disclosure, as utilized herein, when a definition is not otherwise provided, an average particle diameter (D₅₀) indicates a particle size where an accumulated volume is about 50 volume % in a particle size distribution.

In one or more embodiments, the Si-based negative electrode active material or Sn-based negative electrode active material may be utilized by mixing with a carbon-based negative electrode active material. If (e.g., when) a mixture of the Si-based negative electrode active material or Sn-based negative electrode active material and the carbon-based negative electrode active material is utilized, a mixing ratio thereof may be about 1:99 to about 90:10 by weight.

### Negative Electrode

The negative electrode for a rechargeable lithium battery may include a negative electrode current collector and a negative electrode active material layer on the negative electrode current collector. The negative electrode active material layer includes a negative electrode active material and may further include a binder and/or a conductive material.

In one or more embodiments, a content (e.g., amount) of the negative electrode active material may be about 95 wt % to about 99.5 wt % based on 100 wt % of the negative electrode active material layer. A content (e.g., amount) of the binder may be about 0.5 wt % to about 5 wt % based on 100 wt % of the negative electrode active material layer. A content (e.g., amount) of the conductive material may be about 0.5 wt % to about 5 wt % based on 100 wt % of the negative electrode active material layer. For example, in some embodiments, the negative electrode active material layer may include about 90 wt % to about 99 wt % of the negative electrode active material, about 0.5 wt % to about 5 wt % of the binder, and about 0.5 wt % to about 5 wt % of the conductive material.

The binder serves to adhere the negative electrode active material particles to each other and also helps the negative electrode active material to adhere to the negative electrode current collector. The binder may be a non-aqueous binder, an aqueous binder, a dry binder, or a combination thereof.

The non-aqueous binder may include polyvinyl chloride, carboxylated polyvinyl chloride, polyvinyl fluoride, an ethylene propylene copolymer, polystyrene, polyurethane, polytetrafluoroethylene, polyvinylidene fluoride, polyethylene, polypropylene, polyamide-imide, polyimide, or a combination thereof.

The aqueous binder may be selected from a styrene-butadiene rubber, a (meth)acrylated styrene-butadiene rubber, a (meth)acrylonitrile-butadiene rubber, a (meth)acrylic rubber, a butyl rubber, a fluorine rubber, polyethylene oxide, polyvinylpyrrolidone, polyepichlorohydrin, polyphosphazene, poly(meth)acrylonitrile, an ethylene propylene diene copolymer, polyvinylpyridine, chlorosulfonated polyethylene, latex, a polyester resin, a (meth)acrylic resin, a phenol resin, an epoxy resin, polyvinyl alcohol, and a combination thereof.

If (e.g., when) an aqueous binder is utilized as the binder for the negative electrode, the binder may further include a cellulose-based compound capable of imparting viscosity. As the cellulose-based compound, one or more types (kinds) of carboxymethyl cellulose, hydroxypropylmethyl cellulose, methyl cellulose, or an alkali metal salt thereof may be utilized. The alkali metal may be Na, K, or Li.

The dry binder may be a polymer material capable of being fiberized, and may be, for example, polytetrafluoroethylene, polyvinylidene fluoride, a polyvinylidene fluoride-hexafluoropropylene copolymer, polyethylene oxide, or a combination thereof.

The conductive material may be utilized to impart conductivity to the electrode, and in the battery being configured, any electronically conductive material may be utilized as long as it does not cause chemical change. Non-limiting examples thereof may include a carbon-based material such as natural graphite, artificial graphite, carbon black, acetylene black, Ketjen black, a carbon fiber, a carbon nanofiber, and/or a carbon nanotube; a metal-based material including copper, nickel, aluminum, silver, etc. in the form of a metal powder or a metal fiber; a conductive polymer such as a polyphenylene derivative; or a mixture thereof.

The negative electrode current collector may be selected from a copper foil, a nickel foil, a stainless steel foil, a titanium foil, a nickel foam, a copper foam, a polymer substrate coated with a conductive metal, and a combination thereof.

### Separator

In one or more embodiments, the rechargeable lithium battery may further include a separator between the negative electrode and the positive electrode, depending on a type or kind of the rechargeable lithium battery. Such a separator may be, for example, polyethylene, polypropylene, or polyvinylidene fluoride, or multi-layers thereof such as a polyethylene/polypropylene double-layered separator, a polyethylene/polypropylene/polyethylene triple-layered separator, or a polypropylene/polyethylene/polypropylene triple-layered separator.

The separator may include a porous substrate and a coating layer containing an organic material, an inorganic material, or a combination thereof on one or both (e.g., simultaneously) surfaces of the porous substrate.

The porous substrate may be a polymer film formed of any one selected from polyolefin such as polyethylene and/or polypropylene, polyester such as polyethylene terephthalate and/or polybutylene terephthalate, polyacetal, polyamide, polyimide, polycarbonate, polyether ketone, polyaryl ether ketone, polyetherimide, polyamideimide, polybenzimidazole, polyethersulfone, polyphenylene oxide, a cyclic olefin copolymer, polyphenylene sulfide, polyethylene naphthalate, a glass fiber, Teflon, and polytetrafluoroethylene, or a copolymer or a mixture of two or more of them.

The porous substrate may have a thickness of about 1 μm to about 40 μm, for example about 1 μm to about 30 μm, about 1 μm to about 20 μm, about 5 μm to about 15 μm, or about 10 μm to about 15 μm.

The organic material may include a (meth)acryl-based copolymer including a first structural unit derived from (meth)acrylamide, and a second structural unit including at least one selected from among a structural unit derived from (meth)acrylic acid or (meth)acrylate, and a structural unit derived from (meth)acrylamidosulfonic acid or a salt thereof.

The inorganic material may include inorganic particles selected from Al₂O₃, SiO₂, TiO₂, SnO₂, CeO₂, MgO, NiO, CaO, GaO, ZnO, ZrO₂, Y₂O₃, SrTiO₃, BaTiO₃, Mg(OH)₂, boehmite, and a combination thereof, but embodiments of the present disclosure are not limited thereto. An average particle diameter (D₅₀) of the inorganic particles may be about 1 nm to about 2000 nm, for example, about 100 nm to about 1000 nm, or about 100 nm to about 700 nm.

The organic material and the inorganic material may be mixed in one coating layer or may exist in a stacked form of a coating layer including the organic material and a coating layer including the inorganic material.

A thickness of the coating layer may be each about 0.5 μm to about 20 μm, for example, about 1 μm to about 10 μm, or about 1 μm to about 5 μm.

### Rechargeable Lithium Battery

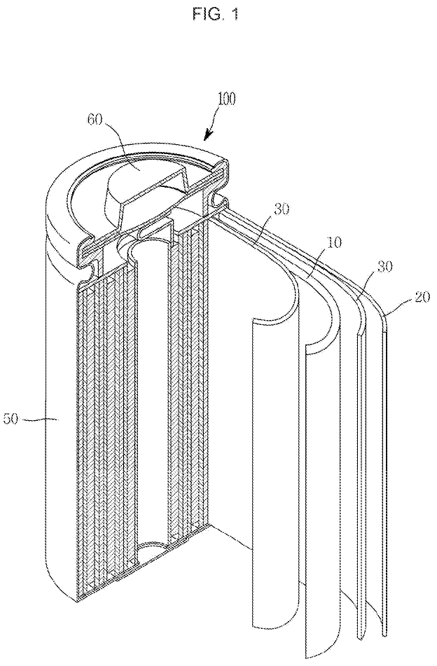

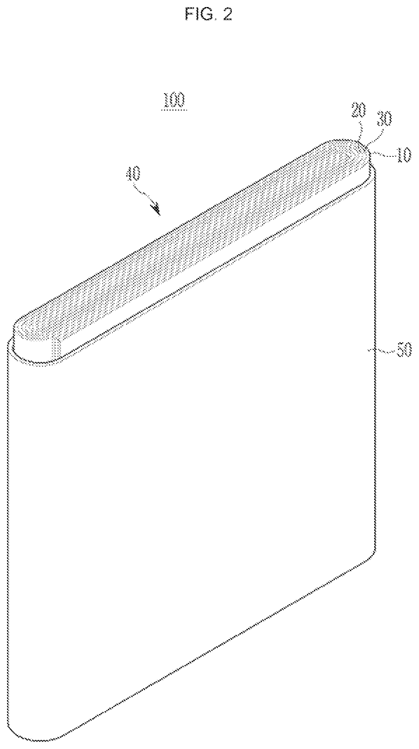

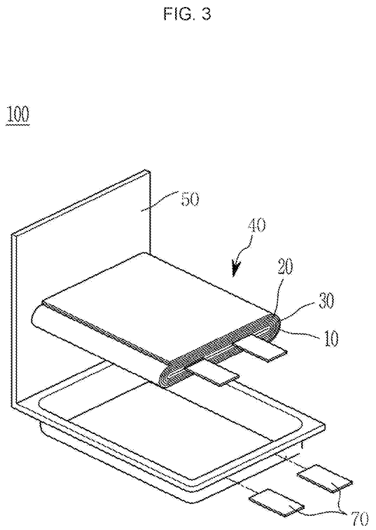

The rechargeable lithium battery may be classified into a cylindrical, prismatic, pouch, or coin battery, etc., depending on a shape thereof. FIGS. 1 to 3 are each of schematic views showing rechargeable lithium batteries according to some embodiments. Referring to FIGS. 1 to 3, in some embodiments, the rechargeable lithium battery 100 may include an electrode assembly 40 including a separator 30 interposed between a positive electrode 10 and a negative electrode 20, and a case 50 in which the electrode assembly 40 is housed. The positive electrode 10, the negative electrode 20, and the separator 30 may be impregnated with an electrolyte. In some embodiments, The rechargeable lithium battery 100 may include a sealing member 60 that seals the case 50 as shown in FIG. 1, and may further include an electrode tab 70 that serves as an electrical path for guiding the current generated in the electrode assembly 40 to the outside as shown in FIG. 3.

Hereinafter, examples and comparative examples of the present disclosure will be described. The following examples are mere examples of the present disclosure, and the present disclosure is not limited to the following examples.

### Synthesis of Additives

### Synthesis Example 1

1,2,4-triazole (2.5 mmol) and K₂CO₃ were added to 200 mL of tetrahydrofuran (THF) and then, stirred for 1 hour at room temperature, and 1,5-dibromopentane (1.1 mmol) was added thereto and then, stirred overnight under reflux.

After distilling the reactant under a reduced pressure, an organic layer alone was separated therefrom in methylene chloride/water, and after removing the methylene chloride, a compound represented by Chemical Formula 1-2 was obtained through a silica column.

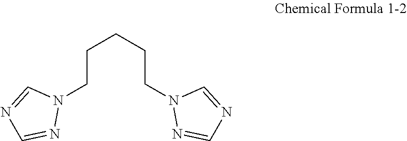 [SMILES: c1ncn(CCCCCn2cncn2)n1]

### Synthesis Example 2

A compound represented by Chemical Formula A was obtained in substantially the same manner as in Synthesis Example 1 except that 1,2,3-benzotriazole (2.5 mmol) and dibromomethane were utilized instead of the 1,2,4-triazole and 1,5-dibromopentane.

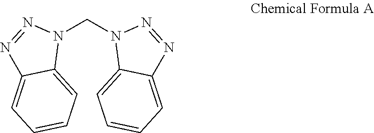 [SMILES: c1ccc2c(c1)nnn2Cn1nnc2ccccc21]

### Synthesis Example 3

A compound represented by Chemical Formula B was obtained in substantially the same manner as in Synthesis Example 1 except that iodine (1.1 mmol) was utilized instead of the 1,5-dibromopentane.

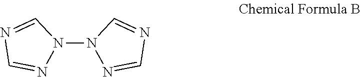

### Preparation of Electrolyte

### Example 1

An electrolyte was prepared by mixing ethylene carbonate (EC), ethylmethyl carbonate (EMC), and dimethyl carbonate (DMC) in a volume ratio of 2:1:7, dissolving 1.5 M LiPF₆ in the mixed non-aqueous organic solvent, and adding 0.05 wt % of the additive represented by Chemical Formula 1-2.

(In the electrolyte composition, a content (e.g., amount) of the additive, “wt %” is based on 100 wt % of the entire electrolyte (lithium salt+non-aqueous organic solvent+additive).)

### Example 2

An electrolyte was prepared in substantially the same manner as in Example 1 except that the additive represented by Chemical Formula 1-2 was added in an amount of 0.1 wt %.

### Example 3

An electrolyte was prepared in substantially the same manner as in Example 1 except that the additive represented by Chemical Formula 1-2 was added in an amount of 0.5 wt %.

### Example 4

An electrolyte was prepared in substantially the same manner as in Example 1 except that the additive represented by Chemical Formula 1-2 was added in an amount of 1.0 wt %.

### Example 5

An electrolyte was prepared in substantially the same manner as in Example 1 except that the additive represented by Chemical Formula 1-2 was added in an amount of 3.0 wt %.

### Comparative Example 1 (Ref.)

An electrolyte was prepared in substantially the same manner as in Example 1 except that no additive was added at all.

### Comparative Example 2

An electrolyte was prepared in substantially the same manner as in Example 1 except that an additive represented by Chemical Formula A was added in an amount of 0.05 wt % instead of the additive represented by Chemical Formula 1-2.

### Comparative Example 3

An electrolyte was prepared in substantially the same manner as in Example 1 except that an additive represented by Chemical Formula B was added in an amount of 0.05 wt % instead of the additive represented by Chemical Formula 1-2.

### Manufacture of Rechargeable Lithium Battery Cells

LiNi_{0.75}Mn_{0.23}Al_{0.02}O₂ as a positive electrode active material, polyvinylidene fluoride as a binder, and acetylene black as a conductive material were mixed respectively in a weight ratio of 96:3:1, and then, dispersed in N-methyl pyrrolidone to prepare a positive electrode active material slurry.

The positive electrode active material slurry was coated on a 15 μm-thick Al foil, dried at 110° C., and pressed to manufacture a positive electrode.

A mixture of artificial graphite and a Si—C composite in a weight ratio of 93:7 was prepared as a negative electrode active material, and the negative electrode active material, a styrene-butadiene rubber binder, and carboxylmethyl cellulose in a weight ratio of 98:1:1 were dispersed in distilled water to prepare a negative electrode active material slurry.

The Si—C composite includes a core including artificial graphite and silicon particles and a coal pitch coated on the surface of the core.

The negative electrode active material slurry was coated on a 10 μm-thick Cu foil, dried at 100° C., and pressed to manufacture a negative electrode.

An electrode assembly was manufactured by assembling the manufactured positive electrode and negative electrode and a 10 μm-thick polyethylene separator, and each rechargeable lithium battery cell was manufactured by injecting the electrolytes of the Examples 1 to 5, and Comparative Examples 1 to 3, respectively.

### Evaluation Examples

The rechargeable lithium battery cells manufactured by respectively utilizing the electrolytes according to Examples 1 to 5 and Comparative Examples 1 to 3 were evaluated as follows, and the results are shown in Tables 1 to 3.

### Evaluation 1: Evaluation of Room-temperature Charge/discharge Cycle Characteristics

The rechargeable lithium battery cells according to Examples 1 to 5 and Comparative Examples 1 to 3 were each charged and discharged under the following conditions to evaluate cycle characteristics, and the results are shown in Table 1.

The cells each were 200 cycles charged and discharged under 0.33 C charge (CC/CV, 4.45 V, 0.025 C Cut-off)/1.0 C discharge (CC, 2.5 V Cut-off) conditions at 25° C. to measure capacity retention and a change in direct current (DC) internal resistance (DC-IR).

The capacity retention was calculated according to Equation 1, and the DC internal resistance change rate was calculated according to Equation 2 based on a voltage changed while discharged by applying a current of SOC 50 C for 30 seconds.

$$Capacity retention rate = ( discharge capacity after 200 cycles / discharge \\ capacity after 1 cycle ) * 100 \qquad Equation 1$$

$$DC internal resistance change rate = \{ ( DC - IR after 200 cycles - DC - IR after 1 cycle ) / \\ ( DC - IR after 1 cycle ) \} * 100 \qquad Equation 2$$

TABLE 1

| | Capacity | Initial | DC-IR after | DC-IR change |
|---|---|---|---|---|
| | retention rate | resistance | 200 cycles | rate after 200 |
| | (@25° C., 200 | @25° C. | @25° C. | cycles |
| | cycles) | (mΩ) | (mΩ) | @25° C. (%) |
| Example 1 | 95.6 | 42.8 | 79.2 | 185 |
| Example 2 | 94.5 | 43.3 | 82.7 | 191 |
| Example 3 | 93.7 | 43.4 | 84.2 | 194 |
| Example 4 | 92.2 | 43.8 | 83.7 | 191 |
| Example 5 | 92.4 | 45.5 | 88.7 | 195 |
| Comparative | 89.2 | 46.1 | 90.4 | 196 |
| Example 1 | | | | |
| Comparative | 88.1 | 44.7 | 87.2 | 195 |
| Example 2 | | | | |
| Comparative | 89.0 | 45.1 | 87.5 | 194 |
| Example 3 | | | | |

Referring to Table 1, when the additive according to one or more embodiments was utilized at room temperature, cycle-life characteristics of the rechargeable lithium battery were improved.

### Evaluation 2: Evaluation of High Temperature (45° C.) Charge/Discharge Cycle Characteristics

The rechargeable lithium battery cells according to Examples 1 to 5 and Comparative Examples 1 to 3 were each charged and discharged under the following conditions to evaluate cycle characteristics, and the results are shown in Table 2.

After 200 cycles charged and discharged under 0.33 C charge (CC/CV, 4.45 V, 0.025 C Cut-off)/1.0 C discharge (CC, 2.5 V Cut-off) condition at 45° C., capacity retention and a change in direct current internal resistance (DC-IR) were measured.

The capacity retention and the DC internal resistance change rate were respectively calculated according to Equation 1 and Equation 2.

TABLE 2

| | | | | DC-IR |
|---|---|---|---|---|
| | Capacity | Initial | DC-IR after | change rate |
| | retention rate | resistance | 200 cycles | after 200 |
| | (@45° C., | @45° C. | @45° C. | cycles |
| | 200 cycles) | (mΩ) | (mΩ) | @45° C. (%) |
| Example 1 | 91 | 47.6 | 106.15 | 223 |
| Example 2 | 89.7 | 49.4 | 115.10 | 233 |
| Example 3 | 89.4 | 49.9 | 119.26 | 239 |
| Example 4 | 88.8 | 49.5 | 117.32 | 237 |
| Example 5 | 89.6 | 50 | 116.00 | 232 |
| Comparative | 84.1 | 50.4 | 120.96 | 240 |
| Example 1 | | | | |
| Comparative | 85 | 50.7 | 123.20 | 243 |
| Example 2 | | | | |
| Comparative | 86.3 | 52.1 | 127.65 | 245 |
| Example 3 | | | | |

Referring to Table 2, when the additive according to one or more embodiments was utilized at a high temperature, not only the cycle-life characteristics of the rechargeable lithium battery were improved, but also an increase in resistance was suppressed or reduced.

### Evaluation 3: Evaluation of High-Temperature Storage Characteristics (Capacity Retention Rate/Capacity Recovery Rate/DC-IR)

Each of the rechargeable lithium battery cells according to Examples 1 to 5 and Comparative Examples 1 to 3 was once charged and discharged at 0.33 C to measure charge and discharge capacity (before the high-temperature storage).

In addition, each of the rechargeable lithium battery cells of Examples 1 to 5 and Comparative Examples 1 to 3 was charged to SOC 100% (a state of being charged to 100% of charge capacity based on 100% of the entire charge capacity of the battery cell), stored at 60° C. for 30 days, and then, discharged to 3.0 V under a constant current condition at 0.33 C to measure initial discharge capacity.

The cells were each recharged to 4.3 V at a constant current of 0.33 C and cut off at an ending current of 0.02 C and discharged to 3.0 V at the constant current of 0.33 C to twice measure discharge capacity. A ratio of first discharge capacity to the initial discharge capacity was calculated as capacity retention (retention capacity), and second discharge capacity was provided as recovery capacity.

The rechargeable lithium battery cells according to Examples 1 to 5 and Comparative Examples 1 to 3 were each measured with respect to initial DC resistance (DC-IR) by calculating ΔV/ΔI (voltage change/current change), and this initial DC resistance (DC-IR) was utilized to calculate a DC-IR increase rate (%) by making the cells into a full-charge state (SOC 100%) as an internal maximum energy state and storing them at a high temperature of 60° C. for 30 days (30D) to measure DC resistance, and the results are shown in Table 3.

$$DC - IR increase rate = ( DC - after 30 days / initial DC - IR ) * 100 \qquad Equation 3$$

TABLE 3

| | Capacity | Capacity | | | |
|---|---|---|---|---|---|
| | retention | recovery | Initial | DC-IR | DC-IR |
| | rate | rate | resistance | @60° C., | change |
| | (@60° C., | (@60° C., | (@25° C., | 30 D | rate |
| | 30 D) | 30 D) | mΩ) | (mΩ) | (%) |
| Example 1 | 88.2 | 89.6 | 42.8 | 100.58 | 235 |
| Example 2 | 86.9 | 89.3 | 43.3 | 104.786 | 242 |
| Example 3 | 85.6 | 87 | 43.4 | 107.632 | 248 |
| Example 4 | 86 | 86.4 | 43.8 | 108.186 | 247 |
| Example 5 | 82.8 | 84.2 | 45.5 | 114.205 | 251 |
| Comparative | 81.3 | 79.7 | 46.1 | 118.938 | 258 |
| Example 1 | | | | | |
| Comparative | 82.2 | 80.6 | 44.7 | 117.114 | 262 |
| Example 2 | | | | | |
| Comparative | 81.5 | 81.9 | 45.1 | 118.613 | 263 |
| Example 3 | | | | | |

Referring to Table 3, the rechargeable lithium battery cells of Examples 1 to 5, compared with the cells of Comparative Examples 1 to 3, each exhibited improved capacity retention and an improved capacity recovery rate during the high-temperature storage but were suppressed or reduced from an increase in resistance.

### SUMMARY

Referring to Tables 1 to 3, Examples 1 to 5 utilizing a compound including triazole itself without a fused benzene ring as an additive, compared with Comparative Example 1 utilizing no additive, exhibited improved high-temperature charge/discharge and storage characteristics of rechargeable lithium battery cells as well as room temperature charge/discharge characteristics thereof.

On the other hand, in Comparative Example 2 utilizing a benzotriazole-based additive, a benzene ring in the corresponding structure was oxidized and decomposed on the positive electrode surface to form an unstable film, thereby increasing resistance of a rechargeable lithium battery cell but decreasing a cycle-life. This phenomenon was more prominent during the high-temperature storage.

In addition, in Comparative Example 3 in which two triazoles were connected by a single bond, because the two triazoles functioned like a monomer rather than a dimer (i.e., only one triazole functioned) and were easily oxidatively decomposed during the high-temperature storage, the resistance of battery increased.

In the present disclosure, when particles are spherical, “diameter” indicates a particle diameter or an average particle diameter, and when the particles are non-spherical, the “diameter” indicates a major axis length or an average major axis length.

A battery management system (BMS) device, and/or any other relevant devices or components according to embodiments of the present invention described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the present disclosure.

While this present disclosure has been described in connection with what is presently considered to be practical example embodiments, it is to be understood that the present disclosure is not limited to the disclosed embodiments, but, on the contrary, is intended to cover one or more suitable modifications and equivalent arrangements included within the spirit and scope of the appended claims and equivalents thereof.

### REFERENCE NUMERALS

100: rechargeable lithium battery
10: positive electrode
20: negative electrode
30: separator
40: electrode assembly
50: case
60: sealing member
70: electrode tab

What is claimed is:

1. An electrolyte additive for a rechargeable lithium battery, the electrolyte additive comprising
an additive represented by Chemical Formula 1: 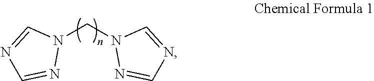
wherein, in Chemical Formula 1,
n is an integer of 1 to 6.

2. The electrolyte additive as claimed in claim 1, wherein n is an integer of 4 to 6.

3. The electrolyte additive as claimed in claim 1, wherein
the additive represented by Chemical Formula 1 is selected from groups:

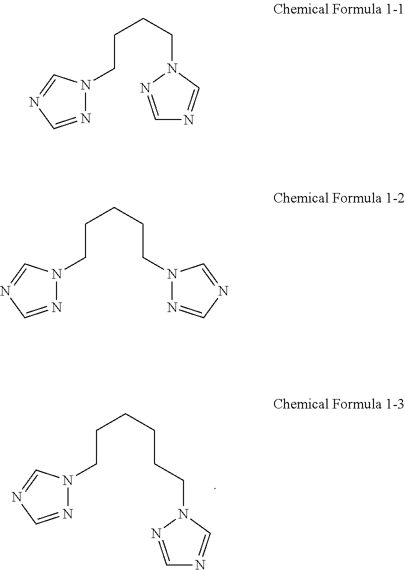 [SMILES: c1ncn(CCCCn2cncn2)n1 ; c1ncn(CCCCCn2cncn2)n1 ; c1ncn(CCCCCCn2cncn2)n1]

4. An electrolyte for a rechargeable lithium battery, the electrolyte comprising
a non-aqueous organic solvent;
a lithium salt; and
an additive represented by Chemical Formula 1: 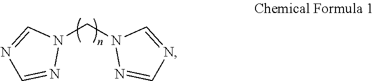
wherein, in Chemical Formula 1,
n is an integer of 1 to 6.

5. The electrolyte as claimed in claim 4, wherein
the additive represented by Chemical Formula 1 is in an amount of about 0.05 wt % to about 3.0 wt % based on a total amount of the electrolyte.

6. The electrolyte as claimed in claim 4, wherein
the non-aqueous organic solvent is a carbonate-based non-aqueous organic solvent.

7. The electrolyte as claimed in claim 4, wherein
the non-aqueous organic solvent comprises ethylene carbonate, ethylmethyl carbonate, dimethyl carbonate, or a combination thereof.

8. The electrolyte as claimed in claim 4, wherein
the lithium salt comprises one or more selected from among LiPF₆, LiBF₄, LiSbF₆, LiAsF₆, LiClO₄, LiAlO₂, LiAlCl₄, LiPO₂F₂, LiCl, Lil, LiN(SO₃C₂F₅)₂, Li(FSO₂)₂N (lithium bis(fluorosulfonyl)imide), LiC₄F₉SO₃, LiN(CₓF₂ₓ₊₁SO₂) (C_{y}F_{2y+1}SO₂) (wherein x and y are each independently an integer from 1 to 20), lithium trifluoromethane sulfonate, lithium tetrafluoroethanesulfonate, lithium difluorobis(oxalato)phosphate, and lithium bis(oxalato) borate.

9. A rechargeable lithium battery, comprising
a positive electrode comprising a positive electrode active material;
a negative electrode comprising a negative electrode active material; and
the electrolyte as claimed in claim 4.

10. The rechargeable lithium battery as claimed in claim 9, wherein
the positive electrode active material is a cobalt-free lithium nickel manganese-based oxide.

11. The rechargeable lithium battery as claimed in claim 10, wherein
the cobalt-free lithium nickel manganese-based oxide comprises a lithium composite oxide represented by Chemical Formula 2:
LiₐNiₓMn_{y}M¹₂M²_{w}O_{2+b}X_{c}, Chemical Formula 2
wherein, in Chemical Formula 2,
0.5<a<1.8,0<b≤0.1,0≤c≤0.1,0≤w<0.1,06≤x<1.0,0<y<0.4, 0<z<0.1, w+x+y+z=1,
M¹ and M² are each independently one or more elements selected from Al, Mg, Ti, Zr, Cr, Sr, V, B, W, Mo, Nb, Si, Ba, Ca, Ce, Cr, and Fe, and
X is one or more elements selected from S, F, P, and Cl.

12. The rechargeable lithium battery as claimed in claim 11, wherein
Chemical Formula 2 is represented by Chemical Formula 2-1:
LiₐNiₓ₁Mn_{y1}Al_{z1}M²_{w1}O_{2+b}X_{c}, and Chemical Formula 2-1
wherein, in Chemical Formula 2-1,
0.5≤a<1.8, 0≤b≤0.1, 0≤c≤0.1, 0≤w1<0.1, 0.6≤x1<1.0, 0<y1<0.4, 0<z1<0.1, w1+x1+y1+z1=1,
M² is one or more elements selected from Mg, Ti, Zr, Cr, Sr, V, B, W, Mo, Nb, Si, Ba, Ca, Ce, Cr, and Fe, and
X is one or more elements selected from S, F, P, and Cl.

13. The rechargeable lithium battery as claimed in claim 12, wherein
in Chemical Formula 2-1, 0.6≤x1≤0.79, 0.2≤y1≤0.39, and 0.01≤z1<0.1.
